(19) United States
(12) Patent Application Publication
(10) Pub. No.: US 2007/0072901 A1
(43) Pub. Date: Mar. 29, 2007

(54) 1-Amino-isoquinoline derivatives for the treatment of diseases associated with inappropriate alk5

(76) Inventor: Yoshiaki Washio, RESEARCH TRIANGLE PARK, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/595,911

(22) PCT Filed: Nov. 17, 2004
(86) PCT No.: PCT/EP04/13072

§ 371 Date: May 19, 2006

(30) Foreign Application Priority Data
Nov. 19, 2003 (GB) 0326963.6

(51) Int. Cl. A61K 31/4709; C07D 401/02

(52) U.S. Cl. 514/307; 546/140; 546/143; 514/308

(57) ABSTRACT

A compound of Formula (I): 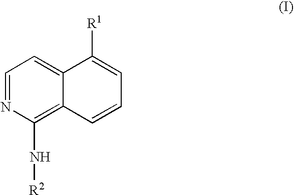 or a salt or solvate thereof,
wherein R¹ represents
   a phenyl or napthyl group, each of which is substituted by one or more substituents independently selected from —OH, —C₁₋₆alkyl, C₁₋₆haloalkyl, —OCH₂OCH₃, —C₁₋₆alkoxy, and -halogen, or
   a mono or bicyclic heteroaryl group comprising 1, 2 or 3 nitrogen atoms, optionally substituted by —C₁₋₆alkoxy, —C₁₋₆alkyl, C₁₋₆haloalkyl or ═O;
R² represents H, benzimidazolyl, benzothiazolyl, piperonyl, isoquinolinyl, quinolinyl, or
   phenyl wherein said phenyl is optionally substituted by —NR³R⁴, —C₁₋₄alkoxy, —C₁₋₆alkyl, —CONR³R⁴, —SO₂NR ³R⁴, —NHCONR³R⁴, —NHCOC₁₋₆alkyl, —C₁₋₆haloalkyl, phenoxy, NH₂ substituted phenoxy, —C₁₋₃alkyl, —C₁₋₃alkoxy, —CF₃, or -5 membered heteroaryl group comprising one or two nitrogen atoms;
R³ and R⁴ are independently selected from H, —C₁₋₆alkyl, —C₁₋₃alkylNR⁵R⁶; and
R⁵ and R⁶ are independently H or C₁₋₃alkyl.

### BACKGROUND OF THE INVENTION

The present invention relates to isoquinoline derivatives, compositions and medicaments containing the same, as well as processes for the preparation and use of such compounds, compositions and medicaments. Such isoquinoline derivatives are potentially useful in the treatment of diseases associated with inappropriate ALK5 activity.

An important large family of enzymes is the protein kinase enzyme family. Currently, there are about 500 different known protein kinases. However, because three to four percent of the human genome is a code for the formation of protein kinases, there may be many thousands of distinct and separate kinases in the human body. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided in two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signaling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix.

Activation of the TGF-β1 axis and expansion of extracellular matrix are early and persistent contributors to the development and progression of chronic renal disease and vascular disease. Border W. A., et al, N. Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818., allograft rejection, HIV nephropathy and angiotensin-induced nephropathy Border W. A., et al, N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991-1003. Thus, inhibition of TGF-β1 activity is indicated as a therapeutic intervention in chronic renal disease.

The present inventors have discovered novel isoquinoline compounds, which are inhibitors of kinase activity, in particular ALK5 activity. Such isoquinoline derivatives are therefore potentially useful in the treatment of disorders associated with inappropriate kinase, more particularly inappropriate ALK5 activity, in particular in the treatment and prevention of various disease states mediated by ALK5 kinase mechanisms, such as chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

### BRIEF SUMMARY OF THE INVENTION

In one aspect of the present invention, there is provided a compound of Formula (I): 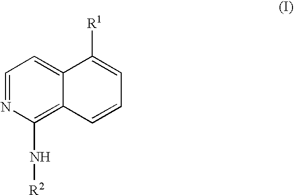
wherein R¹ represents a phenyl or napthyl group (each of which is substituted by one or more substituents independently selected from —OH, —C₁₋₆alkyl, C₁₋₆haloalkyl, —OCH₂OCH₃, —C₁₋₆alkoxy, -halogen,), or a mono or bicyclic heteroaryl group comprising 1, 2 or 3 nitrogen atoms, optionally substituted by —C₁₋₆alkoxy, —C₁₋₆alkyl, C₁₋₆haloalkyl or =0;
R² represents H, benzoimidazolyl, benzothiazolyl, isoquinolinyl, or quinolinyl group or phenyl (said phenyl being optionally substituted by —NR³R⁴, —C₁₋₄alkoxy, —C₁₋₆alkyl, —CONR³R⁴, —SO₂NR³R⁴, —NHCONR³R⁴, —NHCOC₁₋₆alkyl, —C₁₋₆haloalkyl, —OCH₂O—, -phenoxy (wherein the phenyl moiety is optionally substituted by NH₂), —C₁₋₃alkyl, —C₁₋₃alkoxy, —CF₃, -5 membered heteroaryl group comprising one or two nitrogen atoms).
R³ and R⁴ are independently selected from H, —C₁₋₆alkyl, —C₁₋₃alkylNR⁵R⁶;
R⁵ and R⁶ are independently H or C₁₋₃alkyl;
   or a salt, solvate, or physiologically functional derivative thereof:

In a second aspect of the present invention, there is provided a pharmaceutical composition comprising a therapeutically effective amount of a compound of formula (I) or a salt, solvate, or a physiologically functional derivative thereof and one or more of pharmaceutically acceptable carriers, diluents and excipients.

In a third aspect of the present invention, there is provided a compound of formula (I), or a salt, solvate, or a physiologically functional derivative thereof for use in therapy, in particular in the treatment of a disorder mediated by inappropriate ALK5 activity.

In a fourth aspect of the present invention, there is provided a method of treating a disorder in a mammal, said disorder being mediated by inappropriate ALK5 activity, comprising: administering to said mammal a compound of formula (I) or a salt, solvate or a physiologically functional derivative thereof.

In an fifth aspect of the present invention, there is provided the use of a compound of formula (I), or a salt, solvate, or a physiologically functional derivative thereof in the preparation of a medicament for use in the treatment of a disorder mediated by inappropriate ALK5 activity.

In a sixth aspect there is provided a method of treating chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis in a mammal, comprising administering to said mammal a compound of formula (I) or a salt, solvate or a physiologically functional derivative thereof.

In a seventh aspect there is provided a compound of formula (I) or a salt, solvate or physiologically functional derivative thereof in the manufacture of a medicament for the treatment of chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

In an eighth aspect of the invention there is provided a compound of formula (I) or a salt, solvate or physiologically functional derivative thereof for use in the treatment of chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example as used herein, the terms “C₁-C₃ alkyl” and “C₁-C₆ alkyl” refer to an alkyl group, as defined above, containing at least 1, and at most 3 or 6 carbon atoms respectively. Examples of “alkyl” as used herein include, but are not limited to, methyl, ethyl, n-propyl, isopropyl, n-butyl, isobutyl, t-butyl, n-pentyl, isopentyl, n-hexyl and the like.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “C₁-C₆ haloalkyl” refers to an alkyl group as defined above containing the specified number of 6 carbon atoms respectively substituted with at least one halo group, halo being as defined herein. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo.

As used herein, the term “heteroaryl” refers to a monocyclic five to seven membered aromatic ring, or to a fused bicyclic aromatic ring system comprising two of such monocyclic five to seven membered aromatic rings. These heteroaryl rings contain one, two or three nitrogen heteroatoms. Examples of “heteroaryl” groups used herein include pyrrolyl, imidazolyl, pyrazolyl, triazolyl, tetrazolyl, pyridyl, pyridazyl, pyrazinyl, pyrimidyl, quinolinyl, isoquinolinyl, indolyl, indazolyl. The term “5 membered heteroaryl comprising one or two nitrogen atoms” includes pyrrolyl, imidazolyl, pyrazolyl.

As used herein, the term “alkoxy” refers to the group RₐO—, where Rₐ is alkyl as defined above and the terms “C₁-C₄ alkoxy” and “C₁-C₆ alkoxy” refer to an alkoxy group as defined herein wherein the alkyl moiety contains at least 1, and at most 4 or 6, carbon atoms. Exemplary “C₁-C₃ alkoxy” and “C₁-C₆ alkoxy” groups useful in the present invention include, but are not limited to, methoxy, ethoxy, n-propoxy, isopropoxy, n-butoxy, and t-butoxy.

As used herein, the term “haloalkoxy” refers to the group RₐO—, where Rₐ is haloalkyl as defined above and the term “C₁-C₆ haloalkoxy” refers to a haloalkoxy group as defined herein wherein the haloalkyl moiety contains at least 1, and at most 6, carbon atoms. Exemplary C₁-C₆ haloalkoxy groups useful in the present invention include, but is not limited to, trifluoromethoxy.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing, (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

The term “ALK5 inhibitor” is used to mean a compound which inhibits the ALK5 receptor.

The term “ALK5 mediated disease” or a “disorders or diseases mediated by inappropriate ALK5 activity” is used to mean any disease state mediated or modulated by ALK5, kinase mechanisms, in particular chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

As used herein, the term “substituted” refers to substitution with the named substituent or substituents, multiple degrees of substitution being allowed unless otherwise stated.

Preferably R¹ is phenyl (substituted by one or more substituents selected from —OCH₂OCH₃, —OH, -halogen, —OCH₃), naphthyl (substituted by OH), indolinyl, quinolinyl or a pyridinyl moiety (wherein the pyridinyl moiety is optionally substituted by —OCH₃ or ═O).

More preferably R¹ is phenyl substituted by OH. Particularly the OH is on the 5 position of the phenyl ring.

Preferably R² is H, quinolinyl, phenyl (optionally substituted by —SO₂NH₂, CF₃, —CONH₂, -imidazolyl, —OCH₃, C₁₋₃alkyl, —OCH₂O—, CONH CH₂CH₂ N(CH₂CH₃), —O— phenyl (where the phenyl is substituted by NH₂), —NHCOCH₃, NH₂, NHCOCH₃,) or benzoimidazolyl or benzothiazolyl moiety.

More preferably R² is a quinolinyl moiety, particularly a quinoline 6-yl moiety.

While the preferred groups for each variable have generally been listed above separately for each variable, preferred compounds of this invention include those in which several or each variable in Formula (1) is selected from the preferred, more preferred, or most preferred groups for each variable. Therefore, this invention is intended to include all combinations of preferred, more preferred, and most preferred groups.

Specific examples of compounds of the present invention include:
5-(Indol-5-yl)-1-(quinolin-6-yl)aminoisoquinoline;
5-(2-Methoxypyridin-5-yl)-1-(quinolin-6-yl)aminoisoquinoline;
5-(Pyridin-2-on-5-yl)-1-(quinolin-6-yl)aminoisoquinoline;
5-(4-Methoxymethyoxyphenyl)-1-(quinolin-6-yl)aminoisoquinoline;
5-(4-Hydroxyphenyl)-1-(quinolin-6-yl)aminoisoquinoline;
5-(3-Fluoro-4-hydroxyphenyl)-1-(quinolin-6-yl)aminoisoquinoline;
1-Amino-5-(indol-5-yl)isoquinoline;
1-Amino-5-(2-methoxypyridin-5-yl)isoquinoline;
1-Amino-5-(pyridin-2-on-5-yl)isoquinoline;
1-Amino-5-(3-methoxyphenyl)isoquinoline;
5-(2-Hydroxynaphthalen-6-yl)-1-(quinolin-6-yl)aminoisoquinoline;
5-(4Chloro-3-hydroxyphenyl)-1-(quinolin-6-yl)aminoisoquinoline;
3-[5-(4Chloro-3-hydroxyphenyl)-isoquinolin-1-ylamino]benzenesulfonamide;
5-(3-Hydroxyphenyl)-1-(4-trifluoromethylphenyl)aminoisoquinoline;
5-(3-Hydroxyphenyl)-1-(quinolin-6-yl)aminoisoquinoline;
1-(4-Aminocarbonylphenyl)amino-5-(3-hydroxyphenyl)isoquinoline;
5-(3-Hydroxyphenyl)-1-[4-(imidazol-1-yl)phenyl]aminoisoquinoline;
3-[5-(3-Hydroxyphenyl)-isoquinolin-1-ylamino]benzenesulfonamide;
5-(3-Hydroxyphenyl)-1-(3-methoxyphenyl)aminoisoquinoline;
1-(3-Ethylphenyl)amino-5-(3-hydroxyphenyl)isoquinoline;
N-(2-Diethylaminoethyl)-4-[5-(3-hydroxyphenyl)isoquinolin-1-ylamino]benzamide;
1-(3-(4-Aminophenoxy)phenyl)amino-5-(3-hydroxyphenyl)isoquinoline;
5-(3-Hydroxyphenyl)-1-phenylaminoisoquinoline;
5-(3-Hydroxyphenyl)-1-(3,4-methylenedioxyphenyl)aminoisoquinoline;
1-Amino-5-(3-hydroxyphenyl)isoquinoline;
1-(Benzothiazol-6-yl)amino-5-(3-hydroxyphenyl)isoquinoline
1-(Benzimidazol-5-yl)amino-5-(3-hydroxyphenyl)isoquinoline
1-(3-Aminophenyl)amino-5-(3-hydroxyphenyl)isoquinoline
{3-[5-(3-Hydroxyphenyl)isoquinolin-1-ylamino]phenyl}urea
N-{3-[5-(3-Hydroxyphenyl)isoquinolin-1-ylamino]phenyl}acetamide;

It is to be understood that reference to compounds of formula (I) above, following herein, refers to compounds within the scope of formula (I) as defined above with respect to R¹, R², R³, R⁴, R⁵ and R⁶ unless specifically limited otherwise.

The present invention also covers salt of the compounds of formula (I). Typically, the salts of the present invention are pharmaceutically acceptable salts. Salts encompassed within the term “pharmaceutically acceptable salts” refer to non-toxic salts of the compounds of this invention. Suitable salts according to the invention include those formed with both organic and inorganic acids and bases. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

Since the compounds of formula (I) are intended for use in pharmaceutical compositions it will be readily understood that they are each preferably provided in substantially pure form, for example, at least 60% pure, more suitably at least 75% pure and preferably at least 85% pure, especially at least 98% pure (% in a weight for weight basis).

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the composition and not deleterious to the recipient thereof. In accordance with another aspect of the invention there is also provided a process for the preparation of a pharmaceutical composition including admixing a compound of the formula (I), or salts, solvates and physiological functional derivatives thereof, with one or more pharmaceutically acceptable carriers, diluents or excipients.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

Pharmaceutical compositions adapted for vaginal administration may be presented as pessaries, tampons, creams, gels, pastes, foams or spray compositions.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of composition in question, for example those suitable for oral administration may include flavouring agents.

A therapeutically effective amount of a compound of the present invention will depend upon a number of factors including, for example, the age and weight of the animal, the precise condition requiring treatment and its severity, the nature of the composition, and the route of administration, and will ultimately be at the discretion of the attendant physician or veterinarian. However, an effective amount of a compound of formula (I) for the treatment of diseases associated with inappropriate ALK5 activity, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of formula (I) and salts, solvates and physiological functional derivatives thereof, are believed to have utility in chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis as a result of inhibition of the protein kinase ALK5.

The present invention thus also provides compounds of formula (I) and pharmaceutically acceptable salts or solvates thereof, or physiologically functional derivatives thereof, for use in medical therapy, and particularly in the treatment of disorders mediated by ALK5 activity.

The inappropriate ALK5 activity referred to herein is any ALK5 activity that deviates from the normal ALK5 activity expected in a particular mammalian subject. Inappropriate ALK5 activity may take the form of, for instance, an abnormal increase in activity, or an aberration in the timing and or control of ALK5 activity. Such inappropriate activity may result then, for example, from over expression or mutation of the protein kinase leading to inappropriate or uncontrolled activation

The present invention is directed to methods of regulating, modulating, or inhibiting ALK5 for the prevention and/or treatment of disorders related to unregulated ALK5 activity. In particular, the compounds of the present invention can also be used in the treatment of various disease states mediated by ALK5 kinase mechanisms, including chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

A further aspect of the invention provides a method of treatment of a mammal suffering from a disorder mediated by ALK5 activity, which includes administering to said subject an effective amount of a compound of formula (I) or a pharmaceutically acceptable salt, solvate, or a physiologically functional derivative thereof. In a preferred embodiment, the disorder is chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

A further aspect of the present invention provides the use of a compound of formula (I), or a pharmaceutically acceptable salt or solvate thereof, or a physiologically functional derivative thereof, in the preparation of a medicament for the treatment of a disorder characterized by ALK5 activity, in particular, chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

The compound of formula (1) for use in the instant invention and their salts, solvates and physiologically functional derivatives thereof may be used in combination with one or more other therapeutic agents. The invention thus provides in a further aspect the use of a combination comprising a compound of formula (1) with a further therapeutic agent or agents in the treatment of diseases associated with inappropriate ALK5 activity.

When the compounds of formula (1) are used in combination with other therapeutic agents, the compounds may be administered either sequentially or simultaneously by any convenient route.

The combinations referred to above may conveniently be presented for use in the form of a pharmaceutical composition and thus pharmaceutical compositions comprising a combination as defined above optimally together with a pharmaceutically acceptable carrier or excipient comprise a further aspect of the invention. The individual components of such combinations may be administered either sequentially or simultaneously in separate or combined pharmaceutical compositions.

When combined in the same composition it will be appreciated that the two compounds must be stable and compatible with each other and the other components of the composition and may be formulated for administration. When formulated separately they may be provided in any convenient composition, conveniently in such a manner as are known for such compounds in the art.

When a compound of formula (1) is used in combination with a second therapeutic agent active against the same disease, the dose of each compound may differ from that when the compound is used alone. Appropriate doses will be readily appreciated by those skilled in the art.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Those skilled in the art will recognize if a stereocenter exists in compounds of Formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Compounds of Formula I can be prepared according to the synthetic sequences illustrated in Schemes 1, 2, 3 and 4 and further detailed in the Examples section following.

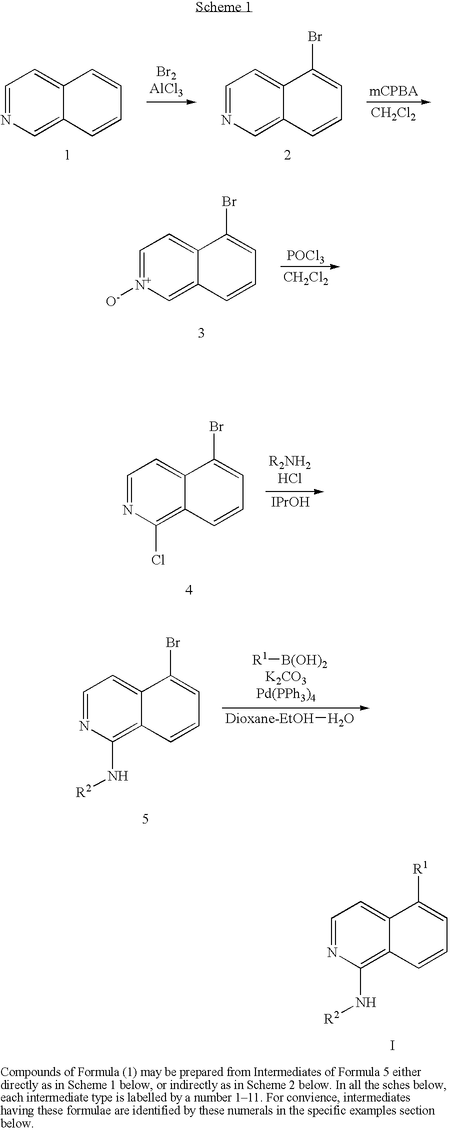

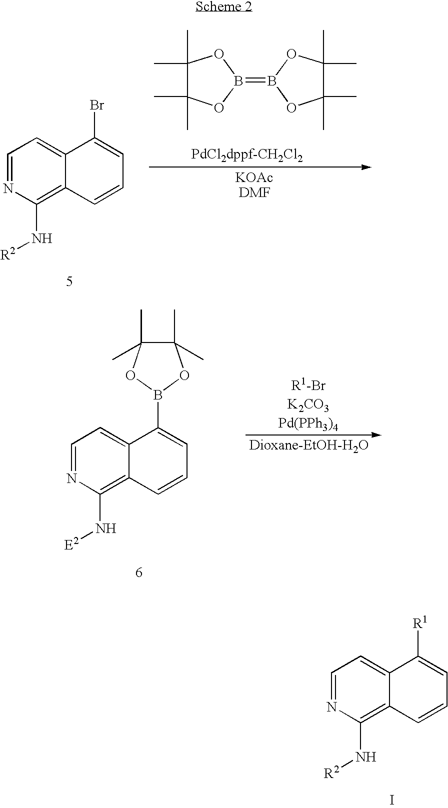

In an alternative method, compounds of Formula (1) wherein R¹ represents -3-hydroxyphenyl may be prepared according to Scheme 3 below:

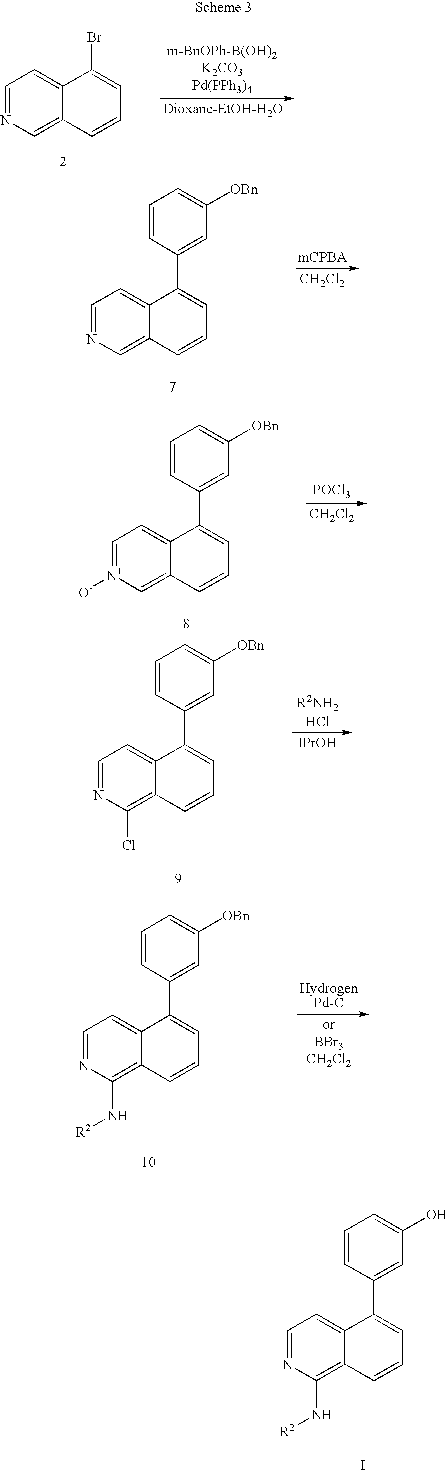

In a further method, when R² is 3-amino phenyl or a phenyl group substituted by NHCOCH₃ or similar amine derivative and R¹ is 3-hydroxyphenyl, Scheme 4 may be employed:

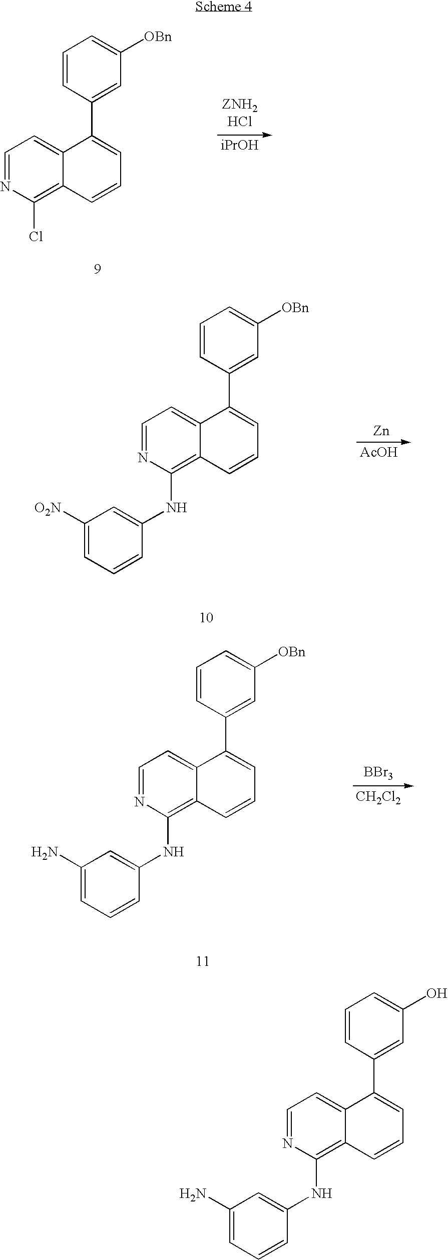

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams);
L (liters);
μL (microliters);
M (molar);
i.v. (intravenous);
MHz (megahertz);
mg (milligrams);
mL (milliliters);
psi (pounds per square inch);
mM (millimolar);
Hz (Hertz);
mol (moles);
mmol (millimoles);
min (minutes);
mp (melting point);
Tᵣ (retention time);
MeOH (methanol);
TEA (triethylamine);
TFAA (trifluoroacetic anhydride);
DMSO (dimethylsulfoxide);
DME (1,2-dimethoxyethane);
DCE (dichloroethane);
DMPU (N,N′-dimethylpropyleneurea);
IBCF (isobutyl chloroformate);
HOSu (N-hydroxysuccinimide);
mCPBA (meta-chloroperbenzoic acid;
EDC (1-[3-dimethylamino)propyl]-3-ethylcarbodiimide hydrochloride);
BOC (tert-butyloxycarbonyl);
DCC (dicyclohexylcarbodiimide);
Ac (acetyl);
TMSE (2-(trimethylsilyl)ethyl);
TIPS (triisopropylsilyl);
DMAP (4-dimethylaminopyridine);
ATP (adenosine triphosphate);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluroniumhexafluoro phosphate).
HEPES (4-(2-hydroxyethyl)-1-piperazine ethane sulfonic acid);
DPPA (diphenylphosphoryl azide);
fHNO₃ (fuming HNO₃); and
rt (room temperature);
h (hours);
TLC (thin layer chromatography);
RP (reverse phase);
i-PrOH (isopropanol);
TFA (trifluoroacetic acid);
THF (trifluoroacetic acid);
AcOEt (ethyl acetate);
DCM (dichloromethane);
DMF (N,N-dimethylformamide);
CDI (1,1-carbonyldiimidazole);
HOAc (acetic acid);
HOBT (1-hydroxybenzotriazole);
FMOC (9-fluorenylmethoxycarbonyl);
DCC (dicyclohexylcarbodiimide);
CBZ (benzyloxycarbonyl);
atm (atmosphere);
TMS (trimethysilyl);
TBS (t-butyldimethylsilyl);
BSA (bovine serum albumin)
HRP (horseradish peroxidase);
EDTA (ethylenediaminetetraacetic acid).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

HPLC were recorded on a Gilson HPLC or Shimazu HPLC system by the following conditions. Column: 50×4.6 mm (id) stainless steel packed with 5 μm Phenomenex Luna C-18 ; Flow rate: 2.0 mL/min; Mobile phase: A phase=50 mM ammonium acetate (pH 7.4), B phase=acetonitrile, 0-0.5 min (A: 100%, B: 0%), 0.5-3.0 min (A: 100-0%, B: 0-100%), 3.0-3.5 min (A: 0%, B: 100%), 3.5-3.7 min (A: 0-100%, B: 100-0%), 3.7-4.5 min (A: 100%, B: 0%); Detection: UV 254 nm; Injection volume: 3 μL.

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Example 1

### 5-(Indol-5-yl)-1-(quinolin-6-yl)aminoisoquinoline

a. 5-Bromoisoquinoline (2)

To a suspension of AlCl₃ (156.7 g, 1.18 mol) in CH₂Cl₂ (500 mL), a solution of isoquinoline (1) (605 mmol, 71 mL) in CH₂Cl₂ (100 mL) was dropwise added at such rate that the reaction mixture was refluxed gently. After addition, CH₂Cl₂ was removed by distillation. The blackish residue was melted at 120° C. then the temperature was adjusted to 100° C. To the mixture, Br₂ (31 mL, 605 mmol) was dropwise added over 2 hrs at 100° C. and stirred for 30 min at same temperature, then was stirred at 75° C. overnight. The mixture was cooled to room temperature then carefully poured into ice-water. The aqueous mixture was basified with NaOHaq. and extracted with ether. The organic layer was dried over Na₂SO₄ then evaporated. Sequence purification on SiO₂ column chromatography twice and recrystallization from hexane gave the title compound (34.5 g, 28%). MS (ESI) (M+H)⁺ 208, 210.

b. 5-Bromoisoquinoline N-oxide (3)

To a solution of 5-bromoisoquinoline (2) (20.8 g, 100 mmol) in CH₂Cl₂ (500 mL), mCPBA (80% assay, 23.7 g, 110 mmol) was added and stirred at 45° C. overnight. After cooling, the mixture was quenched with Na₂S₂O then extracted with CH₂Cl₂. The organic layer was washed with NaOHaq., dried over Na₂SO₄ then evaporated. Sequence recrystallization from CH₂Cl₂-ether gave the title compound (20.1 g, 90%). MS (ESI) (M+H)⁺ 224, 226.

c. 5-Bromo-1-chloroisoquinoline (4)

To a solution of 5-bromoisoquinoline N-oxide (3) (20.1 g, 89.5 mmol) in CH₂Cl₂ (500 mL), POCl₃ (20 mL, 215 mmol) was added and stirred at 45° C. overnight. After cooling, the mixture was evaporated to remove POCl₃ then added water. The mixture was extracted with CH₂Cl₂. The organic layer was washed with NaHCO₃aq., dried over Na₂SO₄ then evaporated. Formed solid was washed with MeOH to give the title compound (16.1 g, 74%). MS (ESI) (M+H)⁺ 242, 244, 246.

d. 5-Bromo-1-(quinolin-6-yl)aminoisoquinoline (5)

To a suspension of 5-bromo-1-chloroisoquinoline (4) (3.0 g, 12.4 mmol) in iPrOH (100 mL), 6-aminoquinoline (4.5 g, 31.2 mmol), 4M HCl-dioxane (5 mL) and MeOH (15 mL) were added and stirred at 80° C. for 3 days. After cooling, the mixture was evaporated to remove solvent then suspended into AcOEt. The mixture was basified with NaHCO₃aq. then formed precipitate was collected by filtration and washed with AcOEt to give the title compound (5) (3.4 g, 78%). MS (ESI) (M+H)⁺ 350, 352.
e. 5-(Indol-5-yl)-1-(quinolin-6-yl)aminoisoquinoline

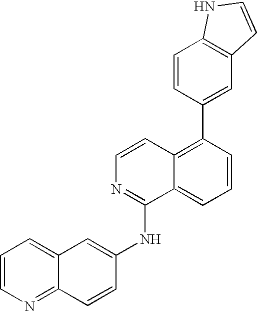

A mixture of 5-bromo-1-(quinolin-6-yl)aminoisoquinoline (5) (70.0 mg, 0.2 mmol), Pd(PPh₃)4 (11.6 mg, 5 mol %) and 5-indole boronic acid (36.5 mg, 0.24 mmol) was flushed N₂ gas then added dioxane (2 mL), ethanol (0.4 mL) and 2M K₂CO₃aq (2 mL). The mixture was stirred at 85° C. overnight. After cooling, the mixture was purified on SCX SPE then SiO₂ column chromatography. Formed solid was washed with MeOH to give the title compound (39.9 mg, 52%).

¹H-NMR (400 MHz, d₆-DMSO), δ 11.26 (s, 1H), 9.57 (s, 1H), 8.74 (dd, 1H), 8.61 (m, 2H), 8.26 (dd, 1H), 8.20 (dd, 1H), 8.04 (d, 1H), 7.97 (d, 1H), 7.72 (m, 2H), 7.64 (s, 1H), 7.55 (d, 1H), 7.45 (m, 2H), 7.20 (m, 2H), and 6.52 (m, 1H); MS (ESI) (M+H)⁺ 387.

### Example 2

### 5-(2-Methoxypyridin-5-yl)-1-(quinolin-6-yl)aminoisoquinoline

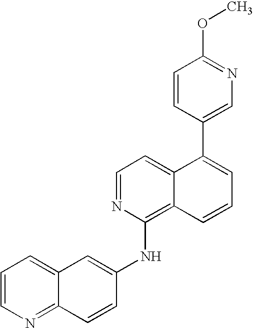

The title compound was prepared from 5-bromo-1-(quinolin-6-yl)aminoisoquinoline (5) and 2-methoxy-5-pyridine boronic acid as described in Example 1e.

¹H-NMR (400 MHz, d₆-DMSO), δ 9.63 (br, 1H), 8.75 (dd, 1H), 8.67 (d, 1H), 8.61 (d, 1H), 8.29 (d, 1H), 8.27 (d, 1H), 8.19 (dd, 1H), 8.09 (d, 1H), 7.98 (d, 1H), 7.88 (dd, 1H), 7.76 (dd, 1H), 7.72 (dd, 1H), 7.47 (dd, 1H), 7.06 (d, 1H), 7.01 (d, 1H), and 3.95 (s, 3H); MS (ESI) (M+H)⁺ 379.

### Example 3

### 5-(Pyridin-2-on-5-yl)-1-(quinolin-6-yl)aminoisoquinoline

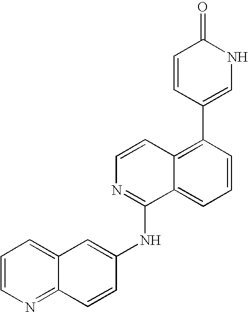

A mixture of 5-(2-methoxypyridin-5-yl)-1-(quinolin-6-yl)aminoisoquinoline (85.3 mg, 0.225 mmol) in conc.HCl aq. (30 mL) was stirred at 50° C. for 3 days then at 100° C. for 1 day. The mixture was evaporated to remove solvent. The residue was passed through NH2-SPE then purified on SCX SPE. Formed solid was washed with CH2Cl2-MeOH to give the title compound (51.9 mg, 63%).

¹H-NMR (400 MHz, d₆-DMSO) δ 11.82 (br, 1H), 9.59 (s, 1H), 8.75 (dd, 1H), 8.62 (m, 2H), 8.27 (d, 1H), 8.19 (dd, 1H), 8.11 (d, 1H), 7.97 (d, 1H), 7.69 (m, 2H), 7.60 (dd, 1H), 7.52 (br, 1H), 7.46 (dd, 1H), 7.13 (d, 1H), and 6.49 (d, 1H); MS (ESI) (M+H)⁺ 365.

### Example 4

### 5-(4-Methoxymethoxyphenyl)-1-(quinolin-6-yl)aminoisoquinoline

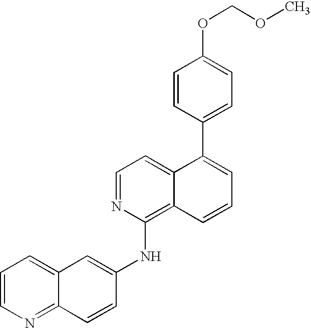

The title compound was prepared from 5-bromo-1-(quinolin-6-yl)aminoisoquinoline (5) and 4-methoxymethyloxyphenyl boronic acid as described in Example 1e.

¹H-NMR (400 MHz, d₆-DMSO), δ 9.59 (br, 1H), 8.74 (dd, 1H), 8.64 (br, 1H), 8.61 (d, 1H), 8.27 (brd, 1H), 8.19 (dd, 1H), 8.07 (d, 1H), 7.97 (d, 1H), 7.73 (dd, 1H), 7.67 (dd, 1H), 7.48-7.43 (m, 3H), 7.20 (d, 2H), 7.13 (d, 1H), 5.29 (s, 2H), and 3.44 (s, 3H); MS (ESI), (M+H)⁺ 408.

### Example 5

### 5-(4-Hydroxyphenyl)-1-(quinolin-6-yl)aminoisoquinoline

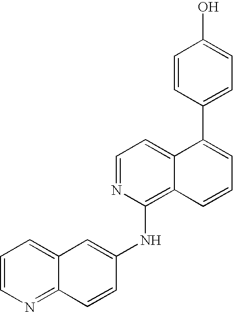

A mixture of 5-(4-Methoxymethyoxyphenyl)-1-(quinolin-6-yl)aminoisoquinoline (68.6 mg, 0.168 mmol) and sat. HCl—MeOH (2mL) in MeOH (12 mL) and water (2 mL) was stirred at 50° C. for 3 days. The mixture was evaporated to remove solvent. The residue was passed through NH₂-SPE then purified on SCX SPE. Formed solid was washed with ether to give the title compound (18.4 mg, 30%).

¹H-NMR (400 MHz, d₆-DMSO), δ 9.56 (s, 1H), 8.74 (dd, 1H), 8.59 (m, 2H), 8.26 (d, 1H), 8.19 (dd, 1H), 8.06 (d, 1H), 7.97 (d, 1H), 7.70 (dd, 1H), 7.62 (d, 1H), 7.45 (dd, 1H), 7.30 (d, 2H), 7.16 (d, 1H), and 6.93 (d, 2H), (1H not detected); MS (ESI), (M+H)⁺ 364.

### Example 6

### 5-(3-Fluoro-4-hydroxyphenyl)-1-(quinolin-6-yl)aminoisoquinoline

a. 5-(4-Benzyloxy-3-fluorophenyl)-1-(quinolin-6-yl)aminoisoquinoline

The title compound was prepared from 5-bromo-1-(quinolin-6-yl)aminoisoquinoline (5) and 4-benzyloxy-3-fluorophenyl boronic acid as described in Example 1e. MS (ESI)m (M+H)⁺ 472.
b. 5-(3-Fluoro-4-hydroxyphenyl)-1-(quinolin-6-yl)aminoisoquinoline

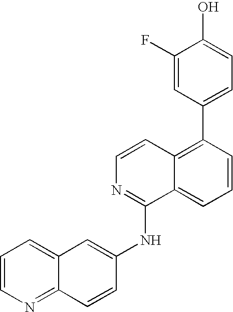

To a mixture of 5-(4-benzyloxy-3-fluorophenyl)-1-(quinolin-6-yl)aminoisoquinoline (116.9 mg, 0.248 mmol) in CH₂Cl₂ (10 mL), BBr₃ in CH₂Cl₂ (1.0M solution, 2.5 mL) was added at −78° C. and was stirred at −78° C. for 3 hrs then at room temperature overnight. The reaction was quenched with methanol then evaporated to remove solvent. The residue was passed through NH₂-SPE then purified on SCX SPE. Formed solid was washed with methanol and ether to give the title compound (57.1 mg, 60%).

¹H-NMR (400 MHz, d₆-DMSO), δ 10.07 (br, 1H), 9.58 (s, 1H), 8.74 (dd, 1H), 8.61 (m, 2H), 8.27 (d, 1H), 8.19 (dd, 1H), 8.08 (d, 1H), 7.97 (d, 1H), 7.71 (dd, 1H), 7.66 (d, 1H), 7.46 (dd, 1H), 7.29 (dd, 1H), and 7.16-7.11 (m, 3H); MS (ESI), (M+H)⁺ 382.

### Example 7

### 1-Amino-5-(indol-5-yl)isoquinoline

a. 1-Amino-5-bromoisoquinoline (5)

A suspension of 5-bromo-1-chloroisoquinoline (4) (2.0 g, 8.25 mmol) in sat. NH₃—MeOH (100 mL) was heated to 180° C. for 15 days in an autoclave. After cooling, the solvent was removed by evaporation. The residue was washed with CH₂Cl₂ then purified on SCX SPE. Formed solid was washed with hexane to give the title compound (1.57 g, 85%). MS (ESI), (M+H)⁺ 223, 225.
b. 1-Amino-5-(indol-5-yl)isoquinoline

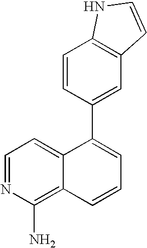

The title compound was prepared from 1-Amino-5-bromoisoquinoline (5) and 5-indole boronic acid as described in Example 1e.

¹H-NMR (400 MHz, d₆-DMSO), δ 11.21 (br, 1H), 8.17 (d, 1H), 7.73 (d, 1H), 7.57 (br, 1H), 7.55 (dd, 1H), 7.52-7.49 (m, 2H), 7.42 (dd, 1H), 7.15 (dd, 1H), 6.85 (d, 1H), 6.78 (br, 2H), and 6.49 (m, 1H); MS (ESI), (M+H)⁺ 260.

### Example 8

### 1-Amino-5-(2-methoxypyridin-5-yl)isoquinoline

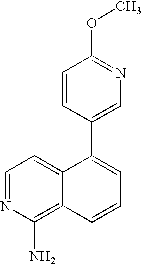

The title compound was prepared from 1-amino-5-bromoisoquinoline (5) and 2-methoxy-5-pyridine boronic acid as described in Example 1e.

¹H-NMR (400 MHz, d₆-DMSO), δ 8.23 (m, 2H), 7.80 (dd, 1H), 7.78 (d, 1H), 7.54 (m, 2H), 6.96 (d, 1H), 6.87 (br, 2H), 6.70 (d, 1H), and 3.93 (s, 3H); MS (ESI), (M+H)⁺ 252.

### Example 9

### 1-Amino-5-(pyridin-2-on-5-yl)isoquinoline

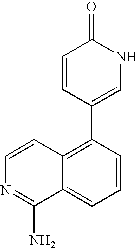

The title compound was prepared from 1-amino-5-(2-methoxypyridin-5-yl)isoquinoline as described in Example 3.

¹H-NMR (400 MHz, d₆-DMSO), δ 11.80 (br, 1H), 8.19 (d, 1H), 7.79 (d, 1H), 7.56-7.43 (m, 4H), 6.85 (br, 2H), 6.76 (d, 1H), and 6.45 (d, 1H); MS (ESI), (M+H)⁺ 238.

### Example 10

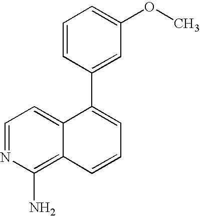

### 1-Amino-5-(3-methoxyphenyl)isoquinoline

The title compound was prepared from 1-amino-5-bromoisoquinoline (5) and 3-methoxyphenyl boronic acid as described in Example 1e.

¹H-NMR (400 MHz, d₆-DMSO), δ 8.21 (d, 1H), 7.76 (d, 1H), 7.53 (m, 2H), 7.42(dd, 1H), 7.03-6.95 (m, 3H), 6.84 (br, 2H), 6.78 (d, 1H), and 3.81 (s, 3H); MS (ESI), (M+H)⁺ 251.

### Example 11

### 5-(2-Hydroxynaphthalen-6-yl)-1-(quinolin-6-yl)aminoisoquinoline

a. 1-(Quinolin-6-yl)amino-5-(4,4,5,5-tetramethyl-[1,3,2]dioxaborolan-2-yl)-isoquinoline (6)

A mixture of 5-bromo-1-(quinolin-6-yl)aminoisoquinoline (5) (350 mg, 1.0 mmol), KOAc (490 mg, 5.0 mmol), PdCl2(dppf)-CH₂Cl₂ (81.7 mg, 10 mol %) and bis(pinacolato)diboron (1.27 g, 5.0 mmol) was flushed N₂ gas then added DMF (8 mL). The mixture was stirred at 80° C. overnight. After cooling, the mixture was purified on SCX SPE to give title compound. (MS (ESI), (M+H)⁺ 398.
b. 5-(2-Hydroxynaphthalen-6-yl)-1-(quinolin-6-yl)aminoisoquinoline

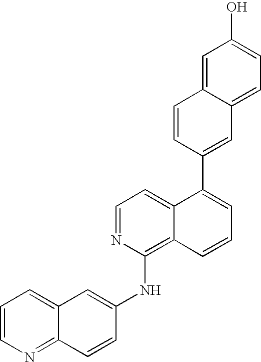

A mixture of 1-(quinolin-6-yl)amino-5-(4,4,5,5-tetramethyl-[1,3,2]dioxaborolan-2-yl)-isoquinoline (6) (37.8 mg, 0.1 mmol), Pd(PPh₃)4 (6.0 mg, 5 mol %) and 2-hydroxy-6-bromonaphthalene (27 mg, 0.12 mmol) was flushed N₂ gas then added dioxane (1.5 mL), ethanol (0.3 mL) and 2M K₂CO₃aq (1.5 mL). The mixture was stirred at 90° C. overnight. After cooling, the mixture was purified on SCX SPE, SiO₂ column chromatography then NH₂ SPE. Formed solid was washed with MeOH to give the title compound (9.5 mg, 23%).

¹H-NMR (400 MHz, d₆-DMSO) δ 9.88 (br, 1H), 9.62 (s, 1H), 8.75 (dd, 1H), 8.67 (dd, 1H), 8.62 (d, 1H), 8.27 (d, 1H), 8.21 (dd, 1H), 8.08 (d, 1H), 7.98 (d, 1H), 7.91 (s, 1H), 7.88 (d, 1H), 7.85 (d, 1H), 7.76 (m, 2H), 7.52 (dd, 1H), 7.47 (dd, 1H), 7.23 (d, 1H), and 7.16 (m, 2H); MS (ESI), (M+H)⁺ 414.

### Example 12

### 5-(4-Chloro-3-hydroxyphenyl)-1-(quinolin-6-yl)aminoisoquinoline

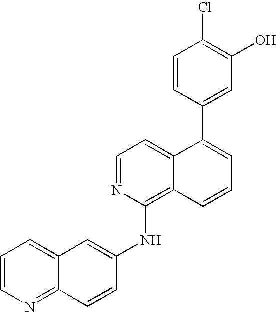

The title compound was prepared from 1-(quinolin-6-yl)amino-5-(4,4,5,5-tetramethyl-[1,3,2]dioxaborolan-2-yl)-isoquinoline (6) and 5-bromo-2-chlorophenol as described in Example 11b.

¹H-NMR (400 MHz, d₆-DMSO), δ 10.45 (br, 1H), 9.61 (s, 1H), 8.75 (dd, 1H), 8.65 (d, 1H), 8.61 (d, 1H), 8.27 (d, 1H), 8.19 (dd, 1H), 8.08 (d, 1H), 7.98 (d, 1H), 7.73 (dd, 1H), 7.66 (d, 1H), 7.47 (m, 2H), 7.13 (d, 1H), 7.76 (d, 1H), and 6.91 (dd, 1H); MS (ESI), (M+H)⁺ 398.

### Example 13

### 3-[5-(4-Chloro-3-hydroxyphenyl)-isoquinolin-1-ylamino]benzenesulfonamide

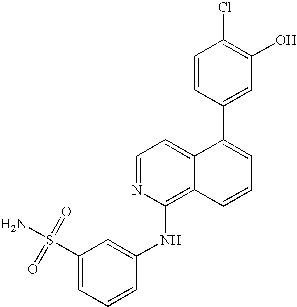 a. 3-(5-Bromoisoquinolin-1-ylamino)benzenesulfonamide (5)

The title compound was prepared from 5-bromo-1-chloroisoquinoline (4) and 3-aminobenzensulfonamide as described in Example 1d. MS (ESI), (M+H)⁺ 378, 380.

b. 3-[5-(4,4,5,5-Tetramethyl-[1,3,2]dioxaborolan-2-yl)-isoquinolin-1-ylamino]benzenesulfonamide (6)

The title compound was prepared from 3-(5-bromoisoquinolin-1-ylamino)benzenesulfonamide (5) as described in Example 11a. MS (ESI), (M−H)⁻ 342 (as boronic acid).

c. 3-[5-(4-Chloro-3-hydroxyphenyl)-isoquinolin-1-ylamino]benzenesulfonamide

The title compound was prepared from 3-[5-(4,4,5,5-tetramethyl-[1,3,2]dioxaborolan-2-yl)-isoquinolin-1-ylamino]benzenesulfonamide (6) and 5-bromo-2-chlorophenol as described in Example 11b.

¹H-NMR (400 MHz, d₆-DMSO), δ 10.38 (br, 1H), 9.57 (s, 1H), 8.60 (d, 1H), 8.43 (dd, 1H), 8.13 (d, 1H), 8.02 (d, 1H), 7.71 (dd, 1H), 7.65 (d, 1H), 7.52 (dd, 1H), 7.48 (dd, 1H), 7.45 (d, 1H), 7.35 (br, 2H), 7.11 (d, 1H), 7.04 (d, 1H), and 6.89 (dd, 1H); MS (ESI), (M+H)⁺ 426.

### Example 14

### 5-(3-Hydroxyphenyl)-1-(4-trifluoromethylphenyl)aminoisoquinoline

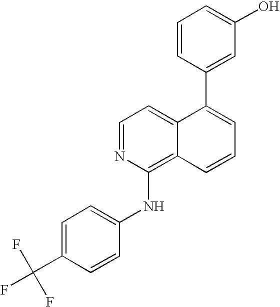 a. 5-(3-Benzyloxyphenyl)isoquinoline (7)

The title compound was prepared from 5-bromoisoquinoline (2) and 3-benzyloxyphenyl boronic acid as described in Example 1e. MS (ESI), (M+H)⁺ 312.

b. 5-(3-Benzyloxyphenyl)isoquinoline N-oxide (8)

The title compound was prepared from 5-(3-benzyloxyphenyl)isoquinoline (7) as described in Example 1b. MS (ESI), (M+H)⁺ 328.

c. 5-(3-Benzyloxyphenyl-1-chloroisoquinoline (9)

The title compound was prepared from 5-(3-benzyloxyphenyl)isoquinoline N-oxide (8) as described in Example 1c. MS (ESI), (M+H)⁺ 346.

d. 5-(3-Benzyloxyphenyl)-1-(4-trifluoromethylphenyl)aminoisoquinoline (10)

The title compound was prepared from 5-(3-benzyloxyphenyl)-1-chloroisoquinoline (9) and 4-trifluoromethylaniline as described in Example 1d. MS (ESI), (M+H)⁺ 471.

e. 5-(3-Hydroxyphenyl)-1-(4-trifluoromethylphenyl)aminoisoquinoline

A mixture of 5-(3-benzyloxyphenyl)-1-(4-trifluoromethylphenyl)aminoisoquinoline (10) (78.4 mg, 0.167 mmol), catalytic amount of 5% Pd—C in methanol (10 mL) was stirred vigorously under hydrogen atmosphere at room temperature for 4 days. The mixture was purified on SCX SPE, SiO₂ column chromatography. Formed solid was washed with ether-hexane to give the title compound (29.1 mg, 46%).

¹H-NMR (400 MHz, d₆-DMSO), δ 9.66 (br, 1H), 9.61 (s, 1H), 8.58 (d, 1H), 8.13 (d, 1H), 8.05 (d, 1H), 7.72 (dd, 1H), 7.68-7.65 (m, 3H), 7.34 (dd, 1H), 7.17 (d, 1H), 6.89 (d, 1H), and 6.86 (m, 2H); MS (ESI) (M+H)⁺ 381.

### Example 15

### 5-(3-Hydroxyphenyl)-1-(quinolin-6-yl)aminoisoquinoline

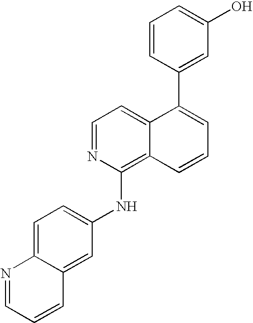

The title compound was prepared from 5-(3-benzyloxyphenyl)-1-chloroisoquinoline (9) and 6-aminoquinoline as described in Example 14d and 14e.

¹H-NMR (400 MHz, d₆-DMSO) δ 9.66 (br, 1H), 9.59 (s, 1H), 8.75 (dd, 1H), 8.64 (d, 1H), 8.61 (d, 1H), 8.27 (dd, 1H), 8.19 (dd, 1H), 8.08 (d, 1H), 7.97 (d, 1H), 7.72 (dd, 1H), 7.66 (d, 1H), 7.47 (dd, 1H), 7.34 (dd, 1H), 7.15 (d, 1H), and 6.90-6.86 (m, 3H); MS (ESI) (M+H)⁺ 364.

### Example 16

### 1-(4-Aminocarbonylphenyl)amino-5-(3-hydroxyphenyl)isoquinoline

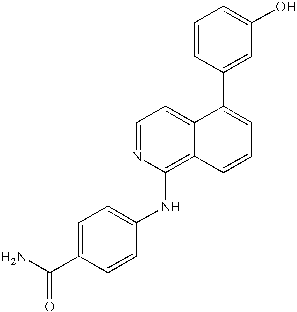

The title compound was prepared from 5-(3-benzyloxyphenyl)-1-chloroisoquinoline (9) and 4-aminobenzene carboxamide as described in Example 14d and 14e.

¹H-NMR (400 MHz, d₆-DMSO) δ 9.64 (s, 1H), 9.50 (br, 1H), 8.57 (d, 1H), 8.03 (d, 1H), 7.97 (d, 2H), 7.86 (d, 2H), 7.83 (br, 1H), 7.71 (dd, 1H), 7.65 (d, 1H), 7.33 (dd, 1H), 7.19 (br, 1H), 7.13 (d, 1H), and 6.89-6.85 (m, 3H); MS (ESI) (M+H)⁺ 356.

### Example 17

### 5-(3-Hydroxyphenyl)-1-[4-(imidazol-1-yl)phenyl]aminoisoquinoline

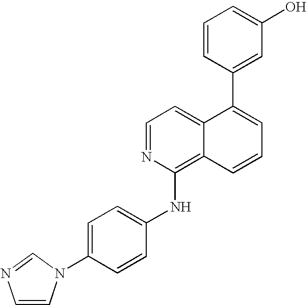

The title compound was prepared from 5-(3-benzyloxyphenyl)-1-chloroisoquinoline (9) and 4-(1-imidazolyl)aniline as described in Example 14d and 14e.

¹H-NMR (400 MHz, d₆-DMSO) δ 9.65 (br, 1H), 9.40 (s, 1H), 8.57 (d, 1H), 8.19 (s, 1H), 8.03 (d, 2H), 7.99 (d, 1H), 7.70 (m, 2H), 7.64 (d, 1H), 7.60 (d, 2H), 7.33 (dd, 1H), 7.09 (m, 2H), and 6.88-6.84 (m, 3H); MS (ESI) (M+H)⁺ 379.

### Example 18

### 3-[5-(3-Hydroxyphenyl)-isoquinolin-1-ylamino]benzenesulfonamide

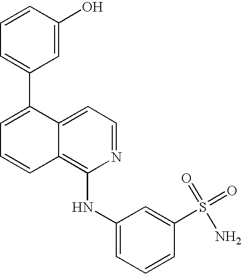

The title compound was prepared from 5-(3-benzyloxyphenyl)-1-chloroisoquinoline (9) and 3-aminobenzene sulfonamide as described in Example 14d and 14e.

¹H-NMR (400 MHz, d₆-DMSO) δ 9.55 (br, 1H), 9.55 (s, 1H), 8.58 (d, 1H), 8.44 (dd, 1H), 8.13 (dd, 1H), 8.01 (dd, 1H), 7.70 (dd, 1H), 7.64 (d, 1H), 7.51 (dd, 1H), 7.44 (m, 1H), 7.34 (m, 3H), 7.12 (d, 1H), and 6.88-6.84 (m, 3H); MS (ESI) (M+H)⁺ 392.

### Example 19

### 5-(3-Hydroxyphenyl)-1-(3-methoxyphenyl)aminoisoquinoline

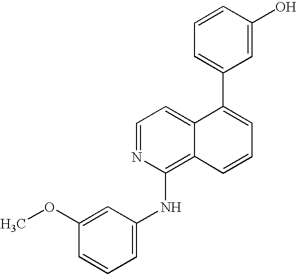

The title compound was prepared from 5-(3-benzyloxyphenyl)-1-chloroisoquinoline (9) and 3-methoxyaniline as described in Example 14d and 14e.

¹H-NMR (400 MHz, d₆-DMSO) δ 9.63 (br, 1H), 9.20 (s, 1H), 8.54 (d, 1H), 7.98 (d, 1H), 7.67 (dd, 1H), 7.61 (m, 2H), 7.50 (dd, 1H), 7.32 (dd, 1H), 7.22 (dd, 1H), 7.06 (d, 1H), 6.88-6.84 (m, 3H), 6.58 (dd, 1H), and 3.77 (s, 3H); MS (ESI) (M+H)⁺ 343.

### Example 20

### 1-(3-Ethylphenyl)amino-5-(3-hydroxyphenyl)isoquinoline

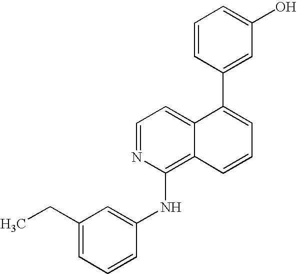

The title compound was prepared from 5-(3-benzyloxyphenyl)-1-chloroisoquinoline (9) and 3-ethylaniline as described in Example 14d and 14e.

¹H-NMR (400 MHz, d₆-DMSO) δ 9.63 (br, 1H), 9.16 (s, 1H), 8.55 (d, 1H), 7.95 (d, 1H), 7.74 (d, 1H), 7.67 (d, 1H), 7.65 (d, 1H), 7.61 (d, 1H), 7.32 (dd, 1H), 7.23 (dd, 1H), 7.03 (d, 1H), 6.87-6.84 (m, 4H), 2.62 (q, 2H), and 1.22 (t, 3H); MS (ESI) (M+H)⁺ 341

### Example 21

### N-(2-Diethylaminoethyl)-4-[5-(3-hydroxyphenyl)isoquinolin-1-ylamino]benzamide

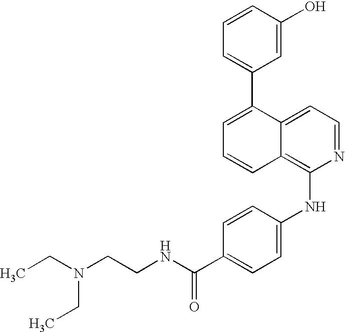

The title compound was prepared from 5-(3-benzyloxyphenyl)-1-chloroisoquinoline (9) and procainamide hydrochloride as described in Example 14d and 14e.

¹H-NMR (400 MHz, d₆-DMSO) δ 9.65 (br, 1H), 9.46 (s, 1H), 8.57 (d, 1H), 8.21 (dd, 1H), 8.03 (d, 1H), 7.97 (d, 2H), 7.81 (d, 2H), 7.71 (dd, 1H), 7.64 (d, 1H), 7.33 (dd, 1H), 7.13 (d, 1H), 6.88-6.85 (m, 3H), and 0.98 (t, 6H), (2H overlapped with H2O, 6H overlapped with DMSO); MS (ESI) (M+H)⁺ 455.

### Example 22

### 1-(3-(4-Aminophenoxy)phenyl)amino-5-(3-hydroxyphenyl)isoquinoline

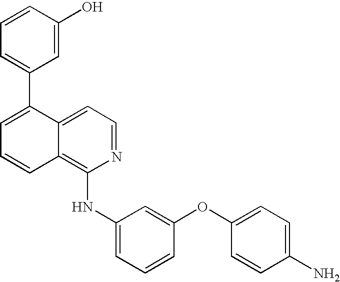

The title compound was prepared from 5-(3-benzyloxyphenyl)-1-chloroisoquinoline (9) and 3-(4-nitrophenyloxy)aniline as described in 14d and 14e.

¹H-NMR (400 MHz, d₆-DMSO) δ 9.63 (br, 1H), 9.23 (s, 1H), 8.51 (d, 1H), 7.95 (d, 1H), 7.65 (dd, 1H), 7.60 (m, 2H), 7.51 (dd, 1H), 7.32 (dd, 1H), 7.21 (dd, 1H), 7.05 (d, 1H), 6.85 (m, 3H), 6.80 (d, 2H), 6.60 (d, 2H), 6.47 (dd, 1H), and 4.96 (br, 2H); MS (ESI) (M+H)⁺ 420.

### Example 23

### 5-(3-Hydroxyphenyl)-1-phenylaminoisoquinoline

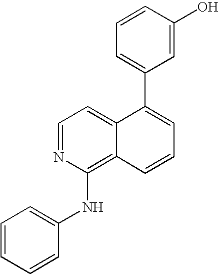

The title compound was prepared from 5-(3-benzyloxyphenyl)-1-chloroisoquinoline (9) and aniline as described in Example 14d and 14e.

¹H-NMR (400 MHz, d₆-DMSO) δ 9.63 (br, 1H), 9.22 (s, 1H), 8.55 (d, 1H), 7.96 (d, 1H), 7.87 (d, 2H), 7.67 (dd, 1H), 7.62 (d, 1H), 7.32 (m, 3H), 7.5 (d, 1H), 7.00 (m, 1H), and 6.88-6.84 (m, 3H); MS (ESI) (M+H)⁺ 312.

### Example 24

### 5-(3-Hydroxyphenyl)-1-(3,4-methylenedioxyphenyl)aminoisoquinoline

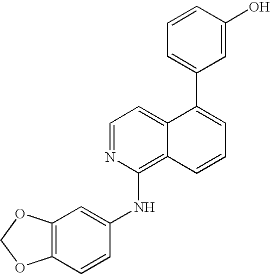

The title compound was prepared from 5-(3-benzyloxyphenyl)-1-chloroisoquinoline (9) and 3,4-methylenedioxyaniline as described in Example 14d and 14e.

¹H-NMR (400 MHz, d₆-DMSO) δ 9.62 (br, 1H), 9.12 (s, 1H), 8.50 (d, 1H), 7.91 (d, 1H), 7.65 (dd, 1H), 7.60 (d, 1H), 7.54 (d, 1H), 7.32 (dd, 1H), 7.23 (dd, 1H), 7.00 (d, 1H), 6.89 (d, 1H), 6.87-6.82 (m, 3H), and 6.00(s, 2H); MS (ESI) (M+H)⁺ 357.

### Example 25

### 1-Amino-5-(3-hydroxyphenyl)isoquinoline

a. 1-Amino-5-(3-benzyloxyphenyl)isoquinoline (10)

The title compound was prepared from 5-(3-benzyloxyphenyl)-1-chloroisoquinoline (9) as described in Example 7a. MS (ESI) (M+H)⁺ 327.
b. 1-Amino-5-(3-hydroxyphenyl)isoquinoline

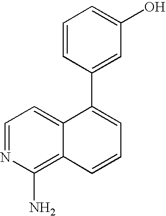

To a mixture of 1-amino-5-(3-benzyloxyphenyl)isoquinoline (10) (54.5 mg, 0.167 mmol) in CH2Cl2 (2 mL), BBr₃ in CH2Cl2 (1.0M solution, 1.7 mL) was added at −78° C. and stirred at −78° C. for 10 min. The temperature was gradually risen to ambient temperature then stirred at room temperature overnight. The reaction was quenched with methanol then evaporated to remove solvent. The residue was purified on SCX SPE. Formed solid was washed with MeOH to give the title compound (21.4 mg, 54%).

¹H-NMR (400 MHz, d₆-DMSO) δ 9.58 (br, 1H), 8.27 (dd, 1H), 7.75 (d, 1H), 7.50 (m, 2H), 7.29 (dd, 1H), and 6.84-6.78 (m, 6H); MS (ESI) (M+H)⁺ 237.

### Example 26

### 1-(Benzothiazol-6-yl)amino-5-(3-hydroxyphenyl)isoquinoline

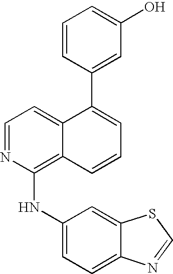

The title compound was prepared from 5-(3-benzyloxyphenyl)-1-chloroisoquinoline (9) and 6-aminobenzothiazole as described in Example 14d and sequence deprotection as described in 25b.

¹H-NMR (400 MHz, d₆-DMSO) δ 9.64 (s, 1H), 9.50 (s, 1H), 9.23 (s, 1H), 8.84 (d, 1H), 8.60 (d, 1H), 8.03 (m, 2H), 7.93 (dd, 1H), 7.71 (dd, 1H), 7.64 (d, 1H), 7.33 (dd, 1H), 7.11 (d, 1H), and 6.90-6.85 (m, 3H); MS (ESI) (M+H)⁺ 370.

### Example 27

### 1-(Benzimidazol-5-yl)amino-5-(3-hydroxyphenyl)isoquinoline

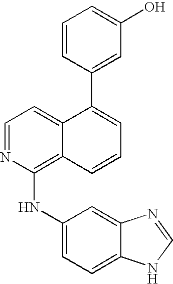

The title compound was prepared from 5-(3-benzyloxyphenyl)-1-chloroisoquinoline (9) and 5-aminobenzimidazole as described in Example 14d and sequence deprotection as described in 25b.

¹H-NMR (400 MHz, d₆-DMSO) δ 12.30 (br, 1H), 9.63 (br, 1H), 9.24 (br, 1H), 8.58 (brd, 1H), 8.30-8.12 (br, 2H), 7.94 (br, 1H), 7.66 (dd, 1H), 7.61-7.50 (br, 3H), 7.32 (dd, 1H), 7.01 (br, 1H), and 6.88-6.83 (m, 3H); MS (ESI) (M+H)⁺ 353.

### Example 28

### 1-(3-Aminophenyl)amino-5-(3-hydroxyphenyl)isoquinoline

a. 5-(3-Benzyloxyphenyl)-1-(3-nitrophenyl)aminoisoquinoline (10)

The title compound was prepared from 5-(3-benzyloxyphenyl)-1-chloroisoquinoline (9) and 3-nitroaniline as described in Example 14d. MS (ESI) (M+H)⁺ 448.

b. 1-(3-Aminophenyl)amino-5-(3-benzyloxyphenyl)isoquinoline (11)

To a solution of 5-(3-benzyloxyphenyl)-1-(3-nitrophenyl)aminoisoquinoline (10) (213.3 mg, 0.477 mmol) in AcOH (10 mL), Zn powder (300 mg) was added and stirred at room temperature for 1.5 hr. The mixture was evaporated to remove solvent. The residue was purified on SCX SPE and NH2 SPE. Sequence crystallization from ether gave the title compound (133.8 mg, 67%). MS (ESI) (M+H)⁺ 418.
c. 1-(3-Aminophenyl)amino-5-(3-hydroxyphenyl)isoquinoline

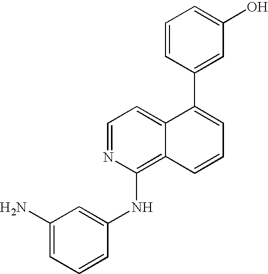

The title compound was prepared from 1-(3-aminophenyl)amino-5-(3-benzyloxyphenyl)isoquinoline (11) as described in 25b.

¹H-NMR (400 MHz, d₆-DMSO) δ 9.64 (br, 1H), 8.94 (s, 1H), 8.52 (dd, 1H), 7.92 (d, 1H), 7.63 (dd, 1H), 7.59 (dd, 1H), 7.32 (dd, 1H), 7.16 (s, 1H), 7.00 (d, 1H), 6.95 (m, 2H), 6.87-6.83 (m, 3H), 6.24 (m, 1H), and 4.98 (d, 2H); MS (ESI) (M+H)⁺ 328.

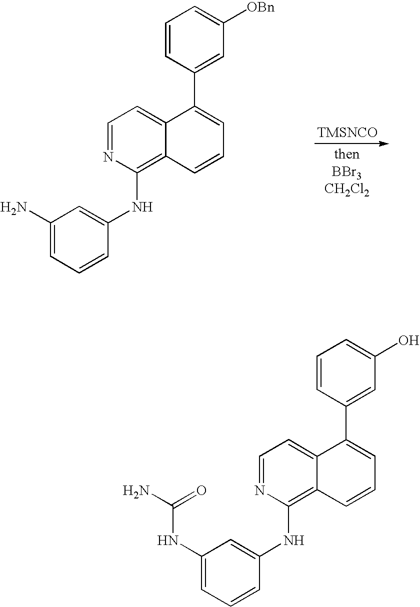

### Example 29

### {3-[5-(3-Hydroxyphenyl)isoquinolin-1-ylamino]phenyl}urea

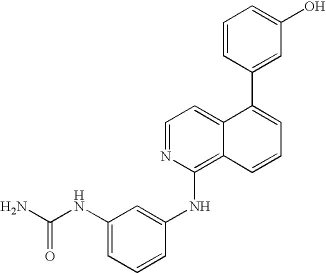

To a mixture of 1-(3-aminophenyl)amino-5-(3-benzyloxyphenyl)isoquinoline (11) (41.8 mg, 0.1 mmol) in THF (1 mL), trimethylsilylisocyanate (14.9 μL, 0.11 mmol) was added and stirred at room temperature for 3 days. The mixture was passed through SCX-SPE to give a crude mixture of {3-[5-(3-benzyloxyphenyl)isoquinolin-1-ylamino]phenyl}urea. The crude mixture was treated with BBr₃ as described in 25b to give the title compound (18.5 mg, 50%).

¹H-NMR (400 MHz, d₆-DMSO) δ 9.63 (br, 1H), 9.17 (s, 1H), 8.56 (d, 1H), 8.48 (s, 1H), 7.95 (d, 1H), 7.89 (dd, 1H), 7.65 (dd, 1H), 7.60 (d, 1H), 7.37 (ddd, 1H), 7.32 (dd, 1H), 7.14 (m, 2H), 7.03 (d, 1H), 6.90-6.84 (m, 3H), and 5.82 (br, 2H); MS (ESI) (M+H)⁺ 371.

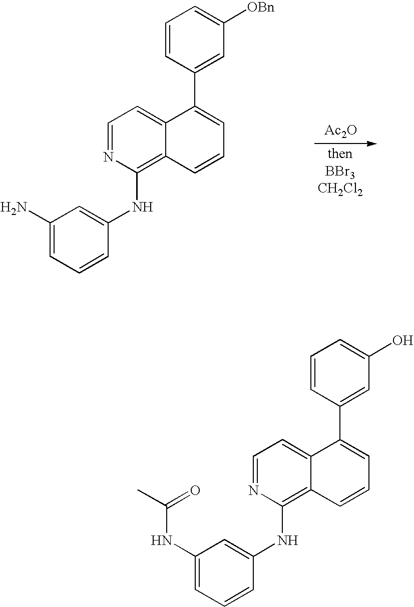

### Example 30

### N-{3-[5-(3-Hydroxyphenyl)isoquinolin-1-ylamino]phenyl}acetamide

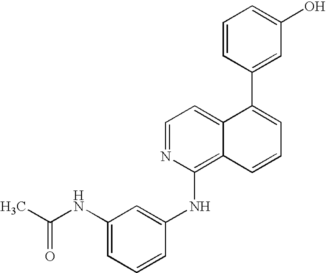

To a mixture of 1-(3-aminophenyl)amino-5-(3-benzyloxyphenyl)isoquinoline (11) (41.8 mg, 0.1 mmol) in THF (1 mL), acetic anhydride (11.3 μL, 0.12 mmol) was added at 0° C. and stirred at room temperature for 3 days. The mixture was passed through SCX-SPE to give a crude mixture of N-{3-[5-(3-benzyloxyphenyl)isoquinolin-1-ylamino]phenyl}acetamide. The crude mixture was treated with BBr₃ as described in 25b to give the title compound (20.9 mg, 57%).

¹H-NMR (400 MHz, d₆-DMSO) δ 9.90 (s, 1H), 9.64 (s, 1H), 9.24 (s, 1H), 8.56 (d, 1H), 8.12 (s, 1H), 7.96 (d, 1H), 7.66 (dd, 1H), 7.61 (dd, 1H), 7.48 (d, 1H), 7.32 (dd, 1H), 7.26 (d, 1H), 7.21 (dd, 1H), 7.05 (d, 1H), 6.88-6.84 (m, 3H), and 2.05 (s, 3H); MS (ESI) (M+H)⁺ 370.

Biological Data

Alk5 Fluorescence Polarization Assay

Assay Principle

Kinase inhibitor compounds, conjugated to fluorophores, can be used as fluorescent ligands to monitor ATP competitive binding of other compounds to a given kinase. The increase in depolarization of plane polarized light, caused by release of the bound ligand into solution, is measured as a polarization/anisotropy value. This protocol details the use of the rhodamine green-labeled ligand depicted below 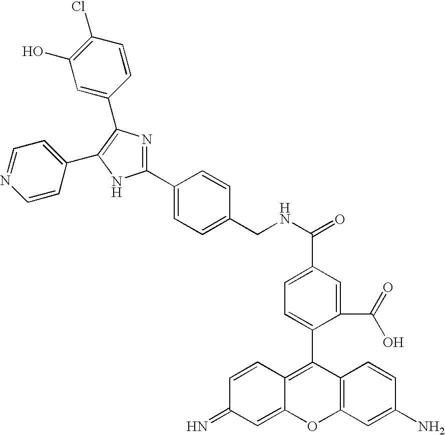
for assays using recombinant GST-ALK5 (residues 198-503). This ligand is derived from 5-[2-(4-aminomethylphenyl)-5-pyridin-4-yl-1H-imidazol-4-yl]-2-chlorophenol and rhodamine green.
Assay Protocol

Assay buffer components: 62.5 mM Hepes pH 7.5 (Sigma H-4034), 1 mM DTT (Sigma D-0632), 12.5 mM MgCl₂ (Sigma M-9272), 1.25 mM CHAPS (Sigma C-3023).

ALK5 was added to assay buffer containing the above components and 1 nM of the fluorescent ligand described above so that the final ALK5 concentration is 10 nM based on active site titration of the enzyme. 40 μl of the enzyme/ligand reagent was added to each well of assay plates containing test compounds. A control compound (1 μl) was added to column 21, rows A-P for the low control values. The plates were read immediately on a LJL Acquest fluorescence reader (Molecular Devices, serial number AQ1048) with excitation, emission, and dichroic filters of 485 nm, 530 nm, and 505 nm, respectively. The fluorescence polarization for each well was calculated by the Acquest and is then imported into curve fitting software for construction of concentration response curves. The percent inhibition of activity was calculated relative to high controls (C1, 1 μl DMSO in column 22, rows A-P)) and low controls (C2, 1 μl of control compound in column 21, rows A-P) using, 100*(1−(U1−C2)/(C1−C2)). The concentration of test compound yielding 50% inhibition was determined using the equation, y=((Vmax*x)/(K+x))+Y2, where “K” was equal to the IC50. The IC50 values were converted to pIC50 values, i.e., −log IC50 in Molar concentration.

All the Exemplified Examples 1-30 were run with the above recited assay and showed inhibitory activity versus ALK5 with a pIC of 5.0 or greater.

What is claimed is:

1. A compound of Formula (I): 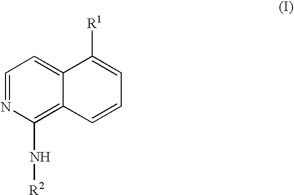
or a salt or solvate thereof,
wherein R¹ represents
a phenyl or napthyl group, each of which is substituted by one or more substituents independently selected from —OH, —C₁₋₆alkyl, C₁₋₆haloalkyl, —OCH₂OCH₃, —C₁₋₆alkoxy, and -halogen, or
a mono or bicyclic heteroaryl group comprising 1, 2 or 3 nitrogen atoms, optionally substituted by —C₁₋₆alkoxy, —C₁₋₆-alkyl, C₁₋₆haloalkyl or ═O;
R² represents
H, benzimidazolyl, benzothiazolyl, piperonyl, isoquinolinyl, quinolinyl, or
phenyl wherein said phenyl is optionally substituted by —NR³R⁴, —C₁₋₄alkoxy, —C₁₋₆alkyl, —CONR³R⁴, —SO₂NR³R⁴, —NHCONR³R⁴, —NHCOC₁₋₆alkyl, —C₁₋₆haloalkyl, phenoxy, NH₂ substituted phenoxy —C₁₋₃alkyl, —C₁₋₃alkoxy, —CF₃, or -5 membered heteroaryl group comprising one or two nitrogen atoms;
R³ and R⁴ are independently selected from H, —C₁₋₆alkyl, —C₁₋₃alkylNR⁵R⁶; and
R⁵ and R⁶ are independently H or C₁₋₃alkyl.

2. A compound according to claim 1 wherein
R¹ is
phenyl substituted by one or more substituents selected from —OCH₂OCH₃, —OH, -halogen, —OCH₃,
OH substituted naphthyl,
indolinyl, quinolinyl, or
a pyridinyl moiety optionally substituted by —OCH₃ or ═O.

3. A compound according to claim 2 wherein R¹ is phenyl substituted by OH.

4. A compound according to claim 3 wherein the OH is substituted meta to the depicted point of attachment of the phenyl to the isoquinoline.

5. A compound according to claim 1 wherein R² is
H, benzimidazolyl, benzothiazolyl, piperonyl, quinoline, or
phenyl wherein said phenyl is optionally substituted by —SO₂NH₂, CF₃, —CONH₂, -imidazolyl, —OCH₃, C₁₋₃ alkyl, CONHCH₂CH₂N(CH₂CH₃)₂, NH2 substituted phenoxy, —NHCOCH₃, NH₂, or NHCOCH₃.

6. A compound according to claim 5 where in R² is a quinoline moiety.

7. A compound according to claim 6 wherein R² is a quinoline 6-yl moiety.

8. A compound as claimed in claim 1, selected from the group consisting of:
5-(Indol-5-yl)-1-(quinolin-6-yl)aminoisoquinoline;
5-(2-Methoxypyridin-5-yl)-1-(quinolin-6-yl)aminoisoquinoline;
5-(Pyridin-2-on-5-yl)-1-(quinolin-6-yl)aminoisoquinoline;
5-(4-Methoxymethyoxyphenyl)-1-(quinolin-6-yl)aminoisoquinoline;
5-(4-Hydroxyphenyl)-1-(quinolin-6-yl)aminoisoquinoline;
5-(3-Fluoro-4-hydroxyphenyl)-1-(quinolin-6-yl)aminoisoquinoline;
1-Amino-5-(indol-5-yl)isoquinoline;
1-Amino-5-(2-methoxypyridin-5-yl)isoquinoline;
1-Amino-5-(pyridin-2-on-5-yl)isoquinoline;
1-Amino-5-(3-methoxyphenyl)isoquinoline;
5-(2-Hydroxynaphthalen-6-yl)-1-(quinolin-6-yl)aminoisoquinoline;
5-(4-Chloro-3-hydroxyphenyl)-1-(quinolin-6-yl)aminoisoquinoline;
3-[5-(4-Chloro-3-hydroxyphenyl)-isoquinolin-1-ylamino]benzenesulfonamide;
5-(3-Hydroxyphenyl)-1-(4-trifluoromethylphenyl)aminoisoquinoline;
5-(3-Hydroxyphenyl)-1-(quinolin-6-yl)aminoisoquinoline;
1-(4-Aminocarbonylphenyl)amino-5-(3-hydroxyphenyl)isoquinoline;
5-(3-Hydroxyphenyl)-1-[4-(imidazol-1-yl)phenyl]aminoisoquinoline;
3-[5-(3-Hydroxyphenyl)-isoquinolin-1-ylamino]benzenesulfonamide;
5-(3-Hydroxyphenyl)-1-(3-methoxyphenyl)aminoisoquinoline;
1-(3-Ethylphenyl)amino-5-(3-hydroxyphenyl)isoquinoline;
N-(2-Diethylaminoethyl)-4-[5-(3-hydroxyphenyl)isoquinolin-1-ylamino]benzamide;
1-(3-(4-Aminophenoxy)phenyl)amino-5-(3-hydroxyphenyl)isoquinoline;
5-(3-Hydroxyphenyl)-1-phenylaminoisoquinoline;
5-(3-Hydroxyphenyl)-1-(3,4-methylenedioxyphenyl)aminoisoquinoline;
1-Amino-5-(3-hydroxyphenyl)isoquinoline;
1-(Benzothiazol-6-yl)amino-5-(3-hydroxyphenyl)isoquinoline;
1-(Benzimidazol-5-yl)amino-5-(3-hydroxyphenyl)isoquinoline;
1-(3-Aminophenyl)amino-5-(3-hydroxyphenyl)isoquinoline;
{3-[5-(3-Hydroxyphenyl)isoquinolin-1-ylamino]phenyl}urea; and
N-{3-[5-(3-Hydroxyphenyl)isoquinolin-1-ylamino]phenyl}acetamide;
or a salt or solvate thereof.

9. A pharmaceutical composition, comprising: a therapeutically effective amount of a compound as claimed in claim 1, or a salt or solvate thereof and one or more of pharmaceutically acceptable carriers, diluents and excipients.

10-12. (canceled)

13. A method of treating a disorder in a mammal, said disorder being mediated by inappropriate ALK5 activity, comprising: administering to said mammal a therapeutically effective amount of a compound as claimed in claim 1, or a salt or solvate thereof.

14. A method according to claim 13 wherein the disorder mediated by inappropriate ALK5 activity is chronic renal disease, acute renal disease, wound healing, photoaging of the skin, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, hepatitis B virus (HBV), hepatitis C virus (HCV), alcohol-induced hepatitis, haemochromatosis and primary biliary cirrhosis, and restenosis.

15-16. (canceled)
